(19) United States
(12) Patent Application Publication
(10) Pub. No.: US 2026/0000452 A1
(43) Pub. Date: Jan. 1, 2026

(54) ACOUSTIC ENERGY DELIVERY ALGORITHM FOR USE WITHIN AN INTRAVASCULAR LITHOTRIPSY DEVICE

(71) Applicant: BOLT MEDICAL, INC., Carlsbad, CA (US)

(72) Inventor: Eric Schultheis, San Clemente, CA (US)

(21) Appl. No.: 18/887,406

(22) Filed: Sep. 17, 2024

Related U.S. Application Data
(60) Provisional application No. 63/616,649, filed on Dec. 31, 2023.

(51) Int. Cl. A61B 18/26; A61B 17/00; A61B 18/00

(52) U.S. Cl. CPC A61B 18/26; A61B 2017/00172; A61B 2017/0019; A61B 2018/0022; A61B 2018/00369; A61B 2018/00642; A61B 2018/00702; A61B 2018/00761; A61B 2018/263; A61B 2018/266

(57) ABSTRACT

A catheter system (100) for treating a treatment site (106) within or adjacent to a vessel wall (108A) of a blood vessel (108), or within or adjacent to a heart valve, includes an energy source (124) and a system controller (126). The energy source (124) generates a plurality of energy pulses. The system controller (126) includes a processor that controls the energy source (124). The system controller (126) is configured to implement an energy control algorithm to control both of (i) a pulse frequency of the plurality of energy pulses, and (ii) an energy level for each of the plurality of energy pulses that are generated by the energy source (124). As the pulse frequency of the plurality of energy pulses increases, the energy level for each of the plurality of energy pulses decreases.

### RELATED APPLICATION

This Application claims priority on U.S. Provisional Patent Application Ser. No. 63/616,649 filed on Dec. 31, 2023, and entitled “ACOUSTIC ENERGY CONTROL SYSTEM AND METHOD FOR USE WITHIN AN INTRAVASCULAR LITHOTRIPSY DEVICE”. To the extent permitted, the contents of U.S. Application Ser. No. 63/616,649 are incorporated in their entirety herein by reference.

### BACKGROUND

Vascular lesions within blood vessels and/or heart valves in the body can be associated with an increased risk for major adverse events, such as myocardial infarction, embolism, deep vein thrombosis, stroke, and the like. Severe vascular lesions can be difficult to treat and achieve patency for a physician in a clinical setting.

Vascular lesions may be treated using interventions such as drug therapy, balloon angioplasty, atherectomy, stent placement, and vascular graft bypass, to name a few. Such interventions may not always be ideal or may require subsequent treatment to address the lesion.

Intravascular lithotripsy is a treatment methodology that has taken on increased importance in recent years for treating vascular lesions within a body of a patient. During an intravascular lithotripsy procedure, high-frequency acoustic waves are generated within a body of a patient. Such high-frequency acoustic waves can cause cavitation to occur, such as in the form of cavitation bubbles, within a catheter fluid inside of a catheter system and/or within the body of the patient. The cavitation bubbles are generated by the rapid change of negative pressure as the acoustic wave travels through the catheter fluid and/or through blood of the patient. After the cavitation bubble is generated, it dissolves back into the catheter fluid and/or blood over time.

However, if the cavitation bubble is not fully dissolved before the next acoustic wave is generated, then the gas nuclei can become a location for subsequent cavitation to occur as the acoustic wave propagates through it. This can result in a much bigger cavitation bubble and/or a rapidly expanding cavitation bubble. Unfortunately, this could lead to undesired outcomes as the cavitation bubble can then reduce and/or dampen the effect of the high-frequency acoustic wave within the catheter system and/or within the body of the patient. Furthermore, if the gas nuclei are present in a vessel wall of an artery, then it could potentially result in unwanted damage to the vessel such as wall rupture, dissections, and trauma.

Accordingly, it is desired to develop an appropriate acoustic energy control system and method (an energy control algorithm) for use within an intravascular lithotripsy device to inhibit the noted problems. More specifically, an appropriate energy control algorithm can be configured to inhibit (i) increased cavitation bubble size that may reduce and/or dampen the effect of the high-frequency acoustic wave within the catheter system and/or within the body of the patient, and (ii) gas nuclei being present within the vessel wall of an artery that may potentially cause unwanted damage to the vessel such as wall rupture, dissections, and trauma.

### SUMMARY

The present invention is directed toward a catheter system for treating a treatment site within or adjacent to a vessel wall of a blood vessel, or within or adjacent to a heart valve. In various embodiments, the catheter system includes an energy source and a system controller. The energy source generates a plurality of energy pulses. The system controller includes a processor that controls the energy source. The system controller is configured to implement an energy control algorithm to control both of (i) a pulse frequency of the plurality of energy pulses, and (ii) an energy level for each of the plurality of energy pulses that are generated by the energy source. In many embodiments, as the pulse frequency of the plurality of energy pulses increases, the energy level for each of the plurality of energy pulses decreases.

In certain embodiments, implementation of the energy control algorithm enables the system controller to automatically control both of (i) the pulse frequency of the plurality of energy pulses, and (ii) the energy level for each of the plurality of energy pulses that are generated by the energy source.

In some embodiments, there is a negative linear relationship between the energy level for each of the plurality of energy pulses and the pulse frequency of the plurality of energy pulses.

In other embodiments, there is a negative non-linear relationship between the energy level for each of the plurality of energy pulses and the pulse frequency of the plurality of energy pulses.

In some embodiments, the energy level of each of the plurality of energy pulses that are generated by the energy source are between approximately 0.05 MPa and 8.0 MPa.

In certain embodiments, the pulse frequency of the plurality of energy pulses that are generated by the energy source are between approximately 0.3 Hz and 15.0 Hz.

In some embodiments, a treatment cycle is defined by a specified number of energy pulses being generated by the energy source over a specified period of time.

In certain embodiments, the energy level of a final energy pulse at an end of the treatment cycle is greater than the energy level of a first energy pulse at a beginning of the treatment cycle.

In some embodiments, the energy control algorithm implemented by the system controller further enables the control system to control at least one of (i) a pulse width of each of the plurality of energy pulses, (ii) a pulse rise time for each of the plurality of energy pulses, (iii) overall acoustic energy per treatment cycle, and (iv) a number of pulses per treatment cycle.

In other embodiments, the energy control algorithm implemented by the system controller further enables the control system to control each of (i) the pulse width of each of the plurality of energy pulses, (ii) the pulse rise time for each of the plurality of energy pulses, (iii) the overall acoustic energy per treatment cycle, and (iv) the number of pulses per treatment cycle.

In many embodiments, the catheter system further includes an energy guide that receives the pulses of energy from the energy source, the energy guide guiding the pulses of energy from a guide proximal end to a guide distal end.

In some embodiments, the catheter system further includes an energy guide that receives the plurality of energy pulses from the energy source, the energy guide guiding the plurality of energy pulses from a guide proximal end to a guide distal end of the energy guide.

In certain embodiments, the catheter system further includes a catheter shaft and a balloon that is coupled to the catheter shaft, the balloon including a balloon wall that defines a balloon interior, the balloon being configured to retain a catheter fluid within the balloon interior.

In some embodiments, the guide distal end of the energy guide is positioned within the balloon interior.

In certain embodiments, the balloon is selectively inflated with the catheter fluid to expand to an inflated state, the balloon wall being positionable adjacent to the treatment site when the balloon is in the inflated state.

In some embodiments, the catheter system further includes a plasma generator that is positioned near the guide distal end of the energy guide, the plurality of energy pulses that are received by the energy guide being emitted from the guide distal end and contacting the plasma generator so that plasma is generated in the catheter fluid retained within the balloon interior.

In certain embodiments, the plasma generation causes bubble formation that generates a pressure wave that imparts pressure adjacent to the treatment site.

In many embodiments, the energy source is a light source that generates a plurality of light energy pulses; and the energy guide is an optical fiber.

The present invention is further directed toward a catheter system for treating a treatment site within or adjacent to a vessel wall of a blood vessel, or within or adjacent to a heart valve, including an energy source that generates a plurality of energy pulses; and a system controller including a processor that controls the energy source, the system controller being configured to implement an energy control algorithm to control both of (i) a pulse frequency of the plurality of energy pulses, and (ii) an energy level for each of the plurality of energy pulses that are generated by the energy source; wherein a treatment cycle is defined by a specified number of energy pulses being generated by the energy source over a specified period of time; and wherein the energy level of a final energy pulse at an end of the treatment cycle is greater than the energy level of a first energy pulse at a beginning of the treatment cycle.

The present invention is also directed toward a catheter system for treating a treatment site within or adjacent to a vessel wall of a blood vessel, or within or adjacent to a heart valve, including an energy source that generates a plurality of energy pulses; an energy guide that receives the plurality of energy pulses from the energy source, the energy guide guiding the plurality of energy pulses from a guide proximal end to a guide distal end of the energy guide; and a system controller including a processor that controls the energy source, the system controller being configured to implement an energy control algorithm to control both of (i) a pulse frequency of the plurality of energy pulses, and (ii) an energy level for each of the plurality of energy pulses that are generated by the energy source; wherein as the pulse frequency of the plurality of energy pulses increases, the energy level for each of the plurality of energy pulses decreases; wherein a treatment cycle is defined by a specified number of energy pulses being generated by the energy source over a specified period of time; wherein the energy level of a final energy pulse at an end of the treatment cycle is greater than the energy level of a first energy pulse at a beginning of the treatment cycle; and wherein the energy control algorithm implemented by the system controller further enables the control system to control each of (i) a pulse width of each of the plurality of energy pulses, (ii) a pulse rise time for each of the plurality of energy pulses, (iii) overall acoustic energy per treatment cycle, and (iv) a number of pulses per treatment cycle.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic cross-sectional view illustration of an embodiment of a catheter system in accordance with various embodiments, the catheter system including a system controller that is configured to implement one or more energy control algorithms having features of the present invention;

FIG. 2 is a graphical illustration of a relationship between acoustic energy level and pulse frequency that can be incorporated into one or more energy control algorithms having features of the present invention;

FIG. 3 is a graphical illustration of another relationship between acoustic energy level and pulse frequency that can be incorporated into one or more energy control algorithms having features of the present invention;

FIG. 4 is a graphical illustration of still another relationship between acoustic energy level and pulse frequency that can be incorporated into one or more energy control algorithms having features of the present invention;

FIG. 5 is a graphical illustration of a relationship between acoustic energy level and pulse count that can be incorporated into one or more energy control algorithms having features of the present invention;

FIG. 6 is a graphical illustration of another relationship between acoustic energy level and pulse count that can be incorporated into one or more energy control algorithms having features of the present invention;

FIG. 7 is a graphical illustration of still another relationship between acoustic energy level and pulse count that can be incorporated into one or more energy control algorithms having features of the present invention;

FIG. 8 is a graphical illustration of yet another relationship between acoustic energy level and pulse count that can be incorporated into one or more energy control algorithms having features of the present invention; and

FIG. 9 is a graphical illustration of still yet another relationship between acoustic energy level and pulse count that can be incorporated into one or more energy control algorithms having features of the present invention.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions at treatment sites within a body of a patient can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

In various embodiments, the catheter systems and related methods disclosed herein can include a balloon catheter configured to advance to a vascular lesion, such as a calcified vascular lesion or a fibrous vascular lesion, at a treatment site located within a body of a patient. In certain implementations, the “treatment site” can be located at or near a vessel wall of a blood vessel of the patient. Additionally, or in the alternative, in other implementations, the “treatment site” can be at or near a heart valve of the patient. Further, or in the alternative, in still other implementations, the “treatment site” can be at another suitable location within the body of the patient.

As defined herein, the terms “treatment site”, “intravascular lesion” and “vascular lesion” may be used interchangeably unless otherwise noted. The intravascular lesions and/or the vascular lesions are sometimes referred to herein as “lesions”.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It is appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is recognized that such a development effort might be complex and time-consuming, but would nevertheless be a routine undertaking of engineering for those of ordinary skill in the art having the benefit of this disclosure.

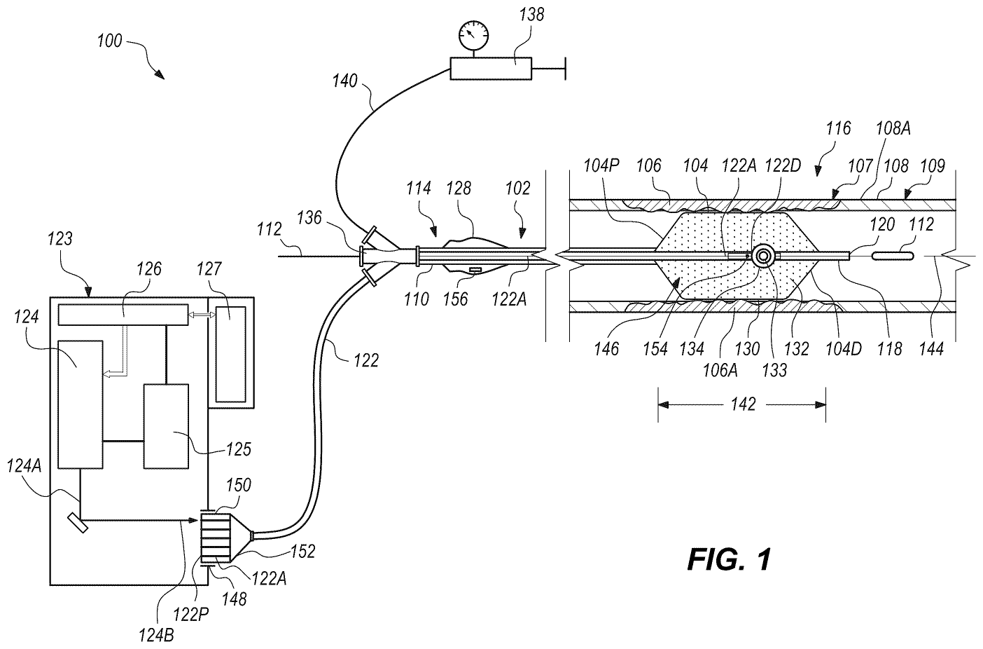

The catheter systems disclosed herein can include many different forms. Referring now to FIG. 1, a simplified schematic cross-sectional view illustration is shown of a catheter system 100 in accordance with various embodiments. The catheter system 100 is suitable for imparting acoustic waves and/or pressure waves to induce fractures at one or more treatment sites 106 within or adjacent to a vessel wall 108A of a blood vessel 108, or on or adjacent to a heart valve, within a body 107 of a patient 109. In the embodiment illustrated in FIG. 1, the catheter system 100 can include (i) a catheter 102 including one or more of an inflatable balloon 104 (sometimes referred to herein simply as a “balloon”), a catheter shaft 110, a guidewire 112, a guidewire lumen 118, an energy guide bundle 122 including one or more energy guides 122A, a source manifold 136, a fluid pump 138, and a handle assembly 128; and (ii) a system console 123 including one or more of an energy source 124, a power source 125, a system controller 126, and a graphic user interface 127 (a “GUI”). Alternatively, the catheter system 100, the catheter 102 and/or the system console 123 can include more components or fewer components than those specifically illustrated and described in relation to FIG. 1.

As illustrated in FIG. 1, the catheter 102 is configured to move to the treatment site 106 within or adjacent to the vessel wall 108A of the blood vessel 108 within the body 107 of the patient 109. The treatment site 106 can include one or more vascular lesions 106A such as calcified vascular lesions, for example. Additionally, or in the alternative, the treatment site 106 can include vascular lesions 106A such as fibrous vascular lesions. Still alternatively, in some implementations, the catheter 102 can be used at a treatment site 106 within or adjacent to a heart valve within the body 107 of the patient 109. Yet alternatively, in certain implementations, the catheter 102 can be used at a treatment site 106 at another suitable location within the body 107 of the patient 109.

As described in detail herein, in various applications, the present invention is directed toward intravascular lithotripsy energy control systems and methods that are tailored to enable the safe breaking apart of vascular lesions 106A within the body 107 of the patient 109, which may include arteries, valve leaflets, and other calcified anatomical structures. More particularly, the system controller 126 within the catheter system 100 can implement the energy control systems and methods described in detail herein (such as in the form of an energy control algorithm that is configured to control output of the energy source 124 in an automated manner) in order to safely break apart vascular lesions 106A within the body 107 of the patient 109, while avoiding the potential drawbacks of (1) increased cavitation bubble size reducing and/or dampening the effect of the high-frequency acoustic wave within the catheter system 100 and/or within the body 107 of the patient 109, and (2) gas nuclei being present within the vessel wall 108A of an artery potentially causing unwanted damage to the vessel such as wall rupture, dissections, and trauma. As further described in detail herein, various embodiments of the energy control algorithm can incorporate preferred parameters relating to one or more of (i) acoustic energy level per energy pulse, (ii) pulse frequency, (iii) pulse width (or pulse duration), (iv) pulse rise time, (v) overall acoustic energy within a given treatment cycle, and (vi) number of energy pulses within a given treatment cycle, and the various interrelationships therebetween. However, it is also appreciated that embodiments of the energy control algorithm can incorporate more specific parameters or fewer specific parameters than those specifically noted herein.

The catheter shaft 110 can extend from a proximal portion 114 of the catheter system 100 to a distal portion 116 of the catheter system 100. The catheter shaft 110 can include a longitudinal axis 144. The guidewire lumen 118 is configured to move over the guidewire 112. As utilized herein, the guidewire lumen 118 defines a conduit through which the guidewire 112 extends. The catheter shaft 110 and/or the catheter 102 can further include an inflation lumen (not shown) and/or various other lumens for various other purposes. In some embodiments, the catheter 102 can have a distal end opening 120 and can accommodate and be tracked over the guidewire 112 as the catheter 102 is moved and positioned at or near the treatment site 106.

The balloon 104 can include a balloon proximal end 104P and a balloon distal end 104D. In certain embodiments, the balloon 104 can be coupled to the catheter shaft 110 and/or to the guidewire lumen 118. More particularly, in some embodiments, the balloon proximal end 104P can be coupled to the catheter shaft 110, and the balloon distal end 104D can be coupled to the guidewire lumen 118.

The balloon 104 includes a balloon wall 130 that defines a balloon interior 146. The balloon 104 can be selectively inflated with a catheter fluid 132 to expand from a deflated state suitable for advancing the catheter 102 through a patient's vasculature, to an inflated state (as shown in FIG. 1) suitable for anchoring the catheter 102 in position relative to the treatment site 106. Stated in another manner, when the balloon 104 is in the inflated state, the balloon wall 130 of the balloon 104 is configured to be positioned substantially directly adjacent to and/or in contact with the treatment site 106.

The balloon 104 suitable for use in the catheter system 100 includes those that can be passed through the vasculature of a patient 109 when in the deflated state. In some embodiments, the balloon 104 is made from silicone. In other embodiments, the balloon 104 can be made from materials such as polydimethylsiloxane (PDMS), polyurethane, polymers such as PEBAX™ material, nylon, or any other suitable material.

The balloon 104 can have any suitable diameter (in the inflated state). In various embodiments, the balloon 104 can have a diameter (in the inflated state) ranging from less than one millimeter (mm) up to 25 mm. In some embodiments, the balloon 104 can have a diameter (in the inflated state) ranging from at least 1.5 mm up to 14 mm. In certain embodiments, the balloon 104 can have a diameter (in the inflated state) ranging from at least two mm up to five mm.

In some embodiments, the balloon 104 can have a length 142 ranging from at least three mm to 300 mm. More particularly, in certain embodiments, the balloon 104 can have a length 142 ranging from at least eight mm to 200 mm. It is appreciated that a balloon 104 having a relatively longer length can be positioned adjacent to larger treatment sites 106, and, thus, may be usable for imparting acoustic waves and/or pressure waves onto and inducing fractures in larger vascular lesions 106A or multiple vascular lesions 106A at precise locations within the treatment site 106. It is further appreciated that a longer balloon 104 can also be positioned adjacent to multiple treatment sites 106 at any one given time.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures of from at least two atm to ten atm.

The balloon 104 can have various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape.

In some embodiments, the balloon 104 can include a drug eluting coating or a drug eluting stent structure. The drug eluting coating or drug eluting stent can include one or more therapeutic agents including anti-inflammatory agents, anti-neoplastic agents, anti-angiogenic agents, and the like.

The catheter fluid 132 used to inflate the balloon 104 can be a liquid or a gas. Some examples of the catheter fluid 132 suitable for use can include, but are not limited to one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the rate of travel of the acoustic waves and/or pressure waves are appropriately manipulated. In certain embodiments, the catheter fluid 132 suitable for use herein is biocompatible. A volume of catheter fluid 132 can be tailored by the chosen energy source 124 and the type of catheter fluid 132 used.

In some embodiments, the contrast agents used in the contrast media can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine-based contrast agents can be used. Suitable non-iodine containing contrast agents can include gadolinium (III)-based contrast agents. Suitable fluorocarbon and perfluorocarbon agents can include, but are not to be limited to, agents such as perfluorocarbon dodecafluoropentane (DDFP, C₅F₁₂).

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the catheter fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium: yttrium-aluminum-garnet (Nd: YAG-emission maximum=1064 nm) lasers, holmium: YAG (Ho: YAG-emission maximum=2.1 μm) lasers, or erbium: YAG (Er: YAG-emission maximum=2.94 μm) lasers. In some embodiments, the absorptive agents can be water-soluble. In other embodiments, the absorptive agents are not water-soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

The catheter shaft 110 of the catheter 102 can be coupled to the one or more energy guides 122A of the energy guide bundle 122 that are in optical communication with the energy source 124. The energy guide(s) 122A can be disposed along the catheter shaft 110 and within the balloon 104. Each of the energy guides 122A can have a guide distal end 122D that is at any suitable longitudinal position relative to the length 142 of the balloon 104 and/or relative to a length of the guidewire lumen 118.

In some embodiments, each energy guide 122A can be an optical fiber and the energy source 124 can be a laser. The energy source 124 can be in optical communication with the energy guides 122A at the proximal portion 114 of the catheter system 100. More particularly, the energy source 124 can selectively, simultaneously, sequentially and/or alternatively be in optical communication with each of the energy guides 122A in any desired combination, sequence and/or pattern.

In some embodiments, the catheter shaft 110 can be coupled to multiple energy guides 122A such as a first energy guide, a second energy guide, a third energy guide, etc., which can be disposed at any suitable positions about and/or relative to the guidewire lumen 118 and/or the catheter shaft 110. For example, in certain non-exclusive embodiments, two energy guides 122A can be spaced apart from one another by approximately 180 degrees about the circumference of the guidewire lumen 118 and/or the catheter shaft 110; three energy guides 122A can be spaced apart from one another by approximately 120 degrees about the circumference of the guidewire lumen 118 and/or the catheter shaft 110; four energy guides 122A can be spaced apart from one another by approximately 90 degrees about the circumference of the guidewire lumen 118 and/or the catheter shaft 110; five energy guides 122A can be spaced apart from one another by approximately 72 degrees about the circumference of the guidewire lumen 118 and/or the catheter shaft 110; six energy guides 122A can be spaced apart from one another by approximately 60 degrees about the circumference of the guidewire lumen 118 and/or the catheter shaft 110; eight energy guides 122A can be spaced apart from one another by approximately 45 degrees about the circumference of the guidewire lumen 118 and/or the catheter shaft 110; or ten energy guides 122A can be spaced apart from one another by approximately 36 degrees about the circumference of the guidewire lumen 118 and/or the catheter shaft 110. Still alternatively, multiple energy guides 122A need not be uniformly spaced apart from one another about the circumference of the guidewire lumen 118 and/or the catheter shaft 110. More particularly, it is further appreciated that the energy guides 122A can be disposed uniformly or non-uniformly about the guidewire lumen 118 and/or the catheter shaft 110 to achieve the desired effect in the desired locations.

In certain embodiments, the guidewire lumen 118 can be substantially annular-shaped and/or cylindrical-shaped and can have a grooved outer surface, with the grooves extending in a generally longitudinal direction along the guidewire lumen 118. In such embodiments, each of the energy guides 122A can be positioned, received and retained within an individual groove formed along and/or into the outer surface of the guidewire lumen 118. Alternatively, the guidewire lumen 118 can be formed without a grooved outer surface, and the position of the energy guides 122A relative to the guidewire lumen 118 can be maintained in another suitable manner.

The catheter system 100, the catheter 102 and/or the energy guide bundle 122 can include any number of energy guides 122A in optical communication with the energy source 124 at the proximal portion 114, and with the catheter fluid 132 within the balloon interior 146 of the balloon 104 at the distal portion 116. For example, in some embodiments, the catheter system 100, the catheter 102 and/or the energy guide bundle 122 can include from one energy guide 122A to greater than 30 energy guides 122A. The guide distal end 122D of each of the energy guides 122A can be at any suitable or desired longitudinal position within the balloon interior 146 relative to the length 142 of the balloon 104. Alternatively, in other embodiments, the catheter system 100, the catheter 102 and/or the energy guide bundle 122 can include greater than 30 energy guides 122A.

The energy guides 122A can have any suitable design that is useful and appropriate for purposes of enabling the generation of plasma, acoustic waves and/or pressure waves in the catheter fluid 132 within the balloon interior 146. Thus, the general description of the energy guides 122A as light guides is not intended to be limiting in any manner, except for as set forth in the claims appended hereto. More particularly, although the catheter systems 100 are often described with the energy source 124 as a light source and the one or more energy guides 122A as light guides, the catheter system 100 can alternatively include any suitable energy source 124 and energy guides 122A for purposes of enabling the generation of the desired plasma in the catheter fluid 132 within the balloon interior 146. For example, in one non-exclusive alternative embodiment, the energy source 124 can be configured to provide high-voltage electrical pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the acoustic waves and/or pressure waves in the catheter fluid 132 that are utilized to provide the fracture force onto the vascular lesions 106A at the treatment site 106. Still alternatively, the energy source 124 and/or the energy guides 122A can have another suitable design and/or configuration, be it electrical, acoustic, pneumatic, other mechanical, etc.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from a guide proximal end 122P toward the guide distal end 122D, with the guide distal end 122D having at least one optical window (not shown) that is positioned within the balloon interior 146.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about the circumference of the guidewire lumen 118 and/or the catheter shaft 110, and the guide distal end 122D of each of the energy guides 122A can be disposed at any suitable longitudinal position relative to the length 142 of the balloon 104 and/or relative to the length of the guidewire lumen 118 to more effectively and more precisely impart acoustic waves and/or pressure waves for purposes of disrupting the vascular lesions 106A at the treatment site 106.

In certain embodiments, the energy guides 122A can include one or more photoacoustic transducers 154, where each photoacoustic transducer 154 can be in optical communication with the energy guide 122A within which it is disposed. In some embodiments, the photoacoustic transducers 154 can be in optical communication with the guide distal end 122D of the energy guide 122A. In such embodiments, the photoacoustic transducers 154 can have a shape that corresponds with and/or conforms to the guide distal end 122D of the energy guide 122A.

The photoacoustic transducer 154 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 154 disposed at the guide distal end 122D of the energy guide 122A can assume the same shape as the guide distal end 122D of the energy guide 122A. For example, in certain non-exclusive embodiments, the photoacoustic transducer 154 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 154 disposed along one or more side surfaces of the length of the energy guide 122A.

In some embodiments, the energy guides 122A can further include one or more diverting structures or “diverters” (not shown in FIG. 1), such as within the energy guide 122A and/or near the guide distal end 122D of the energy guide 122A, that are configured to direct energy from the energy guide 122A toward a side surface which can be located at or near the guide distal end 122D of the energy guide 122A, before the energy is directed toward the balloon wall 130. A diverting structure can include any structure of the system that diverts energy from the energy guide 122A away from its axial path toward a side surface of the energy guide 122A. The energy guides 122A can each include one or more optical windows disposed along the longitudinal or circumferential surfaces of each energy guide 122A and in optical communication with a diverting structure. Stated in another manner, the diverting structures can have any suitable structural configuration that is configured to direct energy in the energy guide 122A toward a side surface that is at or near the guide distal end 122D, where the side surface is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting structures suitable for use include a reflecting element, a refracting element, and a fiber diffuser. The diverting structures suitable for focusing energy away from the guide distal end 122D of the energy guide 122A can include, but are not to be limited to, those having a convex surface, a gradient-index (GRIN) lens, and a mirror focus lens. Upon contact with the diverting structure, the energy can be diverted within the energy guide 122A to one or more of a plasma generating structure 133 (also sometimes referred to as a “plasma generator” or “plasma target”) that is positioned near, but typically spaced apart from, the guide distal end 122D of the energy guide 122A, and the photoacoustic transducer 154 that is in optical communication with a side surface of the energy guide 122A. As referred to herein, the plasma generator 133 can include and/or incorporate any suitable type of structure that is located at or near the guide distal end 122D of the energy guide 122A.

When utilized, the plasma generator 133 receives energy emitted from the guide distal end 122D of the energy guide 122A to generate plasma in the catheter fluid 132 within the balloon interior 146, which, in turn, causes the creation of plasma bubbles (cavitation bubbles) and/or pressure waves that can be directed away from the side surface of the energy guide 122A and toward the balloon wall 130. Additionally, or in the alternative, when utilized, the photoacoustic transducer 154 converts light energy into an acoustic wave that extends away from the side surface of the energy guide 122A.

The source manifold 136 can be positioned at or near the proximal portion 114 of the catheter system 100. The source manifold 136 can include one or more proximal end openings that can receive the one or more energy guides 122A of the energy guide bundle 122, the guidewire 112, and/or an inflation conduit 140 that is coupled in fluid communication with the fluid pump 138. The catheter system 100 can also include the fluid pump 138 that is configured to inflate the balloon 104 with the catheter fluid 132 as needed.

As noted above, in the embodiment illustrated in FIG. 1, the system console 123 includes one or more of the energy source 124, the power source 125, the system controller 126, and the GUI 127. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, and the GUI 127 can be provided within the catheter system 100 without the specific need for the system console 123.

As shown, the system console 123, and the components included therewith, is operatively coupled to the catheter 102, including the energy guide bundle 122, and the remainder of the catheter system 100. For example, in some embodiments, as illustrated in FIG. 1, the system console 123 can include a console connection aperture 148 (also sometimes referred to generally as a “socket”) by which the energy guide bundle 122 is mechanically coupled to the system console 123. In such embodiments, the energy guide bundle 122 can include a guide coupling housing 150 (which can generally include one or more ferrules) that houses a portion, such as at least the guide proximal end 122P, of each of the energy guides 122A. At least a portion of the guide coupling housing 150 is configured to fit and be selectively retained within the console connection aperture 148 to provide the mechanical coupling between the energy guide bundle 122 and the system console 123, as well as helping to provide an optical coupling between the energy source 124 and the energy guides 122A of the energy guide bundle 122.

The energy guide bundle 122 can also include a guide bundler 152 (or “shell”) that brings each of the individual energy guides 122A closer together so that the energy guides 122A and/or the energy guide bundle 122 can be in a more compact form as it extends as part of the catheter 102 into the blood vessel 108 or the heart valve during use of the catheter system 100.

The energy source 124 can be selectively and/or alternatively coupled in optical communication with each of the energy guides 122A, such as to the guide proximal end 122P of each of the energy guides 122A, in the energy guide bundle 122. In particular, the energy source 124 is configured to generate energy in the form of a source beam 124A, such as a pulsed source beam, that can be selectively and/or alternatively directed to and received by each of the energy guides 122A in the energy guide bundle 122, such as through the use of a multiplexer (not shown), as an individual guide beam 124B. Alternatively, the catheter system 100 can include more than one energy source 124. For example, in one non-exclusive alternative embodiment, the catheter system 100 can include a separate energy source 124 for each of the energy guides 122A in the energy guide bundle 122.

The energy source 124 can have any suitable design. In certain embodiments, the energy source 124 can be configured to provide sub-millisecond pulses of energy from the energy source 124 that are focused onto a small spot in order to couple it into the guide proximal end 122P of the energy guide 122A. Such pulses of energy are then directed and/or guided along the energy guides 122A to a location within the balloon interior 146 of the balloon 104, thereby inducing plasma formation in the catheter fluid 132 within the balloon interior 146 of the balloon 104, such as via the plasma generator 133 that can include and/or incorporate any suitable structure that is located at or near the guide distal end 122D of the energy guide 122A.

In many embodiments, the plasma generator 133 can be positioned slightly spaced apart from the guide distal end 122D of the energy guide 122A. In certain embodiments, the plasma generator 133 can be provided in the form of a backstop-type structure with an angled face that redirects energy emitted from the guide distal end 122D toward the balloon wall 130 of the balloon 104 and/or toward the vessel wall 108A of the blood vessel 108 at the treatment site 106. Alternatively, the plasma generator 133 can have another suitable structural design.

In particular, the energy emitted at the guide distal end 122D of the energy guide 122A is directed toward, contacts and energizes material of the plasma generator 133, such as material on an angled face of the plasma generator 133, for purposes of generating plasma in the catheter fluid 132 within the balloon interior 146. The plasma generation ionizes and superheats the surrounding catheter fluid 132 and thus causes rapid inertial bubble formation, and imparts pressure waves upon the treatment site 106. An exemplary plasma-induced bubble 134 is illustrated in FIG. 1.

The plasma generator 133 can be formed from any suitable materials. For example, in certain non-exclusive embodiments, the plasma generator 133 can be formed from one or more metals such as titanium, stainless steel, tungsten, tantalum, platinum, molybdenum, niobium, iridium, etc. Alternatively, the plasma generator 133 may be formed from plastics such as polyimide and nylon. Still alternatively, the plasma generator 133 can be formed from other suitable materials.

It is appreciated that the energy source 124 can be controlled by the system controller 126 such that the pulses of energy from the energy source 124 exhibit any desired parameters. More specifically, as described herein, the system controller 126 can control the energy source 124, in an automated manner, through implementation of a specially-designed energy control algorithm such that the pulses of energy generated by the energy source 124 have any desired pulse frequency, energy level, pulse width, and/or pulse rise time. The system controller 126 can further control the energy source 124 through use of the energy control algorithm such that the pulses of energy from the energy source 124 are grouped together with any desired number of pulses within a given treatment cycle and/or with any desired overall energy level per treatment cycle. As referred to herein, a “treatment cycle” can encompass a number of pulses of energy that are delivered to the treatment site 106 over a specified duration of time. Alternatively, the system controller 126 can control the energy source 124 such that the pulses of energy from the energy source 124 exhibit certain desired parameters other than those specifically noted above.

In various non-exclusive alternative embodiments, the sub-millisecond pulses of energy from the energy source 124 can be delivered to the treatment site 106 at a pulse frequency of between approximately one hertz (Hz) and 5000 Hz, between approximately 30 Hz and 1000 Hz, between approximately ten Hz and 100 Hz, or between approximately one Hz and 30 Hz. Alternatively, the sub-millisecond pulses of energy can be delivered to the treatment site 106 at a pulse frequency that can be greater than 5000 Hz or less than one Hz, or any other suitable range of pulse frequencies.

It is appreciated that although the energy source 124 is typically utilized to provide pulses of energy, the energy source 124 can still be described as providing a single source beam 124A, i.e. a single pulsed source beam.

The energy sources 124 suitable for use can include various types of light sources including lasers and lamps. For example, in certain non-exclusive embodiments, the energy source 124 can be an infrared laser that emits energy in the form of pulses of infrared light. Alternatively, as noted above, the energy sources 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (μs) timescales. It is appreciated that there are many combinations of laser wavelengths, pulse widths and energy levels that can be employed to achieve plasma and the associated cavitation bubbles in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

Exemplary nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 KHz.

In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

In still other embodiments, the energy source 124 can include a plurality of lasers that are grouped together in series. In yet other embodiments, the energy source 124 can include one or more low energy lasers that are fed into a high energy amplifier, such as a master oscillator power amplifier (MOPA). In still yet other embodiments, the energy source 124 can include a plurality of lasers that can be combined in parallel or in series to provide the energy needed to create the plasma bubble 134 (or cavitation bubble) in the catheter fluid 132.

The catheter system 100 can generate acoustic waves and/or pressure waves having maximum pressures in the range of at least one megapascal (MPa) to 100 MPa. The maximum pressure generated by a particular catheter system 100 will depend on the energy source 124 and the various parameters of the energy pulses from the energy source, the absorbing material, the bubble expansion, the propagation medium, the balloon material, and other factors. In various non-exclusive alternative embodiments, the catheter systems 100 can generate acoustic waves and/or pressure waves having maximum pressures in the range of at least approximately 0.1 MPa to 50 MPa, at least approximately 0.1 MPa to 30 MPa, or at least approximately 15 MPa to 25 MPa.

The acoustic waves and/or pressure waves can be imparted upon the treatment site 106 from a distance within a range from at least approximately 0.1 millimeters (mm) to greater than approximately 25 mm extending radially from the energy guides 122A when the catheter 102 is placed at the treatment site 106. In various non-exclusive alternative embodiments, the acoustic waves and/or pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending radially from the energy guides 122A when the catheter 102 is placed at the treatment site 106. In other embodiments, the acoustic waves and/or pressure waves can be imparted upon the treatment site 106 from another suitable distance that is different than the foregoing ranges. In some embodiments, the acoustic waves and/or pressure waves can be imparted upon the treatment site 106 within a range of at least approximately 0.1 MPa to 30 MPa at a distance from at least approximately 0.1 mm to ten mm. In some embodiments, the acoustic waves and/or pressure waves can be imparted upon the treatment site 106 from a range of at least approximately 0.1 MPa to 25 MPa at a distance from at least approximately 0.1 mm to ten mm. Still alternatively, other suitable pressure ranges and distances can be used.

The power source 125 is electrically coupled to and is configured to provide necessary power to each of the energy source 124, the system controller 126, the GUI 127, and the handle assembly 128. The power source 125 can have any suitable design for such purposes.

The system controller 126 is electrically coupled to and receives power from the power source 125. The system controller 126 is coupled to and is configured to control operation of each of the energy source 124 and the GUI 127. The system controller 126 can include one or more processors or circuits for purposes of controlling the operation of at least the energy source 124 and the GUI 127. For example, the system controller 126 can control the energy source 124, in an automated manner, through use and/or implementation of an energy control algorithm for purposes of generating pulses of energy as desired and/or at any desired firing rate or frequency of pulse delivery. The system controller 126 can further be configured to control the energy source 124 for generating pulses of energy with any desired energy level, with any desired pulse width or pulse duration, with any desired rise time, with any suitable number of pulses per treatment cycle, and/or with any other desired suitable energy parameters. Stated in another manner, the energy control algorithms of the present invention, as used and/or implemented through the system controller 126, can be configured to control the energy source 124 to output energy pulses having any desired pulse frequency, pulse energy level, pulse width (or pulse duration), pulse rise time, and/or overall energy output and pulse number for a given treatment cycle.

More specifically, as described in greater detail herein below, the system controller 126 can be configured to implement one or more energy control algorithms, including desired combinations of energy pulse frequency and desired energy level, so as to inhibit the above-noted problems of increased cavitation bubble size reducing and/or dampening the effect of the high-frequency acoustic wave within the catheter system 100 and/or within the body 107 of the patient 109, and gas nuclei being present within the vessel wall 108A of an artery potentially causing unwanted damage to the blood vessel 108 such as wall rupture, dissections, and trauma. In addition to the noted energy pulse frequency and the pulse energy level, in certain implementations, the energy control algorithms can further factor in such variables as pulse width (or pulse duration), pulse rise time, number of pulses per treatment cycle, overall energy per treatment cycle, and/or any other desired suitable energy parameters.

As further described herein, it is appreciated that the size of the cavitation bubble (or plasma bubble 134) is proportional to the energy and signature of the acoustic wave. For instance, an acoustic wave that has a small peak-to-peak pressure signature will result in a smaller cavitation bubble than that of an acoustic wave that has a larger peak-to-peak signature. Thus, an acoustic wave having a small peak-to-peak pressure signature, which generates a corresponding smaller cavitation bubble, can be combined with a somewhat greater pulse frequency, while still inhibiting the undesired outcomes noted above. This is because the smaller cavitation bubbles may dissolve more quickly back into the catheter fluid 132 and/or the blood of the patient 109 than a larger cavitation bubble.

Other factors, such as rise time, acoustic pulse width, and overall acoustic energy, can also contribute to changes in the cavitation event. Thus, any energy control algorithm implemented through the system controller 126 should take into consideration any potential increases or decreases in pulse rise time, pulse width, and overall energy level when exercising the desired control of the energy source 124 and the energy pulses being generated therefrom.

During intravascular lithotripsy therapy, multiple pulses of acoustic energy are delivered to the treatment site 106 over a duration of time to achieve a treatment cycle. The frequency of the pulse delivery, and number of pulses can be adjusted for each treatment cycle. In certain clinical situations, such as with intravascular lithotripsy balloon therapy in the coronary arteries, it is desirable for the treatment cycle time to be as short as possible to minimize vessel occlusion within the heart. Therefore, it is optimal for frequency of pulse delivery to be relatively fast in that situation. However, as described above, if the frequency of the pulse delivery is too fast for the cavitation bubble to fully dissolve before the next acoustic wave is delivered, then certain unwanted performance and safety effects, such as described above, can occur. Thus, the user may want to change the frequency of pulse delivery during the procedure, and an energy control algorithm that (automatically) modifies the acoustic parameters as described above to reduce the size of the cavitation bubble and therefore reducing the potential for gas build-up can be employed.

In summary, in accordance with various embodiments of the present invention, the energy control algorithms that are implemented through use of the system controller 126 can include various energy parameters, such as one or more of (1) pulse frequency, (2) energy level per pulse, (3) pulse width (or pulse duration), (4) pulse rise time, (5) overall acoustic energy per treatment cycle, and (6) number of pulses per treatment cycle. It is appreciated that any and all of the above-noted energy parameters can be utilized and combined in any suitable manner through implementation of the energy control algorithms to provide improved efficacy for the catheter system 100 in an automated manner. It is further appreciated, however, that the energy control algorithms that are implemented through use of the system controller 126 can also include other parameters or variables of the energy pulses from the energy source 124 than those specifically listed above.

Certain features and aspects of particular energy control systems and methods (energy control algorithms) will be illustrated and described in greater detail herein below.

The system controller 126 can further be configured to control operation of other components or aspects of the catheter system 100, such as the positioning of the catheter 102 and/or the guide distal end 122D of the energy guides 122A adjacent to the treatment site 106, the inflation of the balloon 104 with the catheter fluid 132, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 128.

The GUI 127 is accessible by the user or operator of the catheter system 100. The GUI 127 is electrically connected to the system controller 126. With such design, the GUI 127 can be used by the user or operator to ensure that the catheter system 100 is effectively utilized to impart pressure onto and induce fractures into the vascular lesions 106A at the treatment site 106. The GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during use of the catheter system 100. In various embodiments, the GUI 127 can include one or more colors, different sizes, varying brightness, etc., that may act as alerts to the user or operator. Additionally, or in the alternative, the GUI 127 can provide audio data or information to the user or operator. The specifics of the GUI 127 can vary depending upon the design requirements of the catheter system 100, or the specific needs, specifications and/or desires of the user or operator.

As shown in FIG. 1, the handle assembly 128 can be positioned at or near the proximal portion 114 of the catheter system 100, and/or near the source manifold 136. In this embodiment, the handle assembly 128 is coupled to the balloon 104 and is positioned spaced apart from the balloon 104. Alternatively, the handle assembly 128 can be positioned at another suitable location.

The handle assembly 128 is attached to the catheter shaft 110 and is handled and used by the user or operator to operate, position and control the catheter 102. The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the energy source 124, the fluid pump 138, and the GUI 127.

In some embodiments, the handle assembly 128 can integrate and/or include at least a portion of the system controller 126 within an interior of the handle assembly 128. For example, as shown, in certain embodiments, the handle assembly 128 can include circuitry 156, which is electrically coupled between catheter electronics and the system console 123, and which can form at least a portion of the system controller 126. In some embodiments, the circuitry 156 can transmit such electrical signals or otherwise provide data to the system controller 126.

In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 128, such as within the system console 123. It is understood that the handle assembly 128 can include fewer or additional components than those specifically illustrated and described herein.

As with all embodiments illustrated and described herein, various structures may be omitted from the figures for clarity and ease of understanding. Further, the figures may include certain structures that can be omitted without deviating from the intent and scope of the invention.

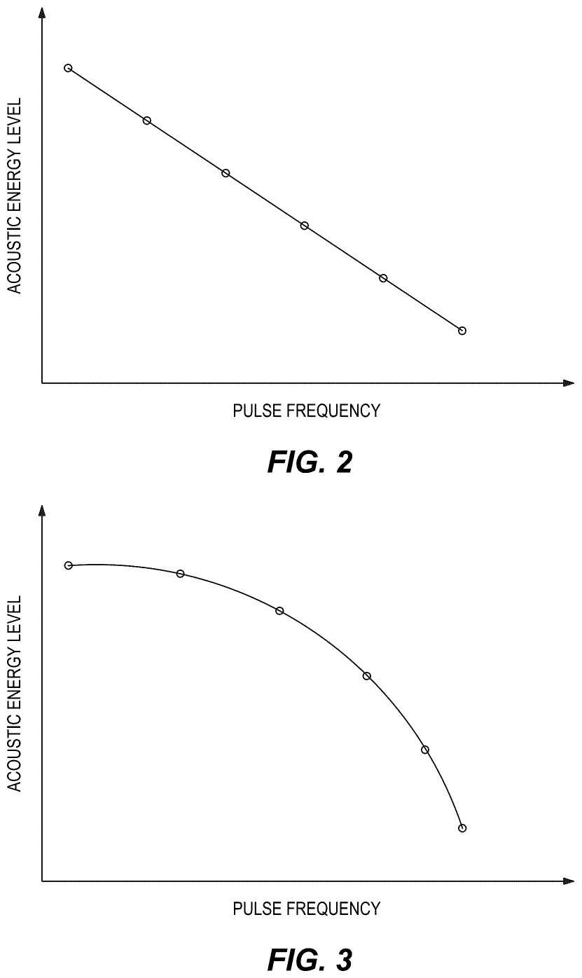

The following FIGS. 2-9 illustrate certain features and aspects that can be incorporated into one or more energy control algorithms that can be implemented through use of the system controller in order to avoid the undesired consequences of (i) increased cavitation bubble size reducing and/or dampening the effect of the high-frequency acoustic wave within the catheter system and/or within the body of the patient; and (ii) gas nuclei being present within the vessel wall of an artery potentially causing unwanted damage to the vessel such as wall rupture, dissections, and trauma. As noted, however, it is further appreciated that the energy control algorithms can also incorporate many additional energy parameters other than those specifically noted in FIGS. 2-9. Thus, the energy parameters specifically referred to in FIGS. 2-9 are not intended to be limiting in any manner as to the various energy parameters that may ultimately be included within the energy control algorithms being implemented as part of the present invention.

FIG. 2 is a graphical illustration of a relationship between acoustic energy level and pulse frequency that can be incorporated into one or more energy control algorithms having features of the present invention. It is appreciated that the energy control algorithms implemented by the system controller can utilize any suitable relationships between the acoustic energy level and the pulse frequency of the energy pulses generated by the energy source, as each of the acoustic energy level and the pulse frequency can be increased and/or decreased (or maintained at a substantially constant level) in any suitable manner. However, as described herein, certain relationships are believed to be generally more preferable for purposes of inhibiting the undesired consequences described above.

As noted above, the size of the cavitation bubble is proportional to the energy and signature of the acoustic wave. For example, an acoustic wave having a small peak-to-peak pressure signature typically results in a smaller cavitation bubble than that of an acoustic wave having a larger peak-to-peak signature. Other factors, such as rise time, acoustic pulse width, overall acoustic energy, can also contribute to changes in the cavitation event.

As further noted above, during a given intravascular lithotripsy therapy procedure, pulses of acoustic energy are delivered to the treatment site over a duration of time as part of a treatment cycle. The pulse frequency, as well as the total number of energy pulses, can be modified as desired for each treatment cycle. For some intravascular therapy procedures, such as such therapy procedures in the coronary arteries, it is generally preferred that the treatment cycle be relatively short so as to minimize vessel occlusion within the heart. Therefore, pulse frequency will typically be faster for such procedures. However, it is further appreciated that if the pulse frequency is too fast for the cavitation bubble to fully dissolve before the next acoustic wave is delivered, then certain detrimental performance and safety effects, such as described above, can occur. Thus, the operator may want to adjust the pulse frequency during the procedure by implementing one or more energy control algorithms that modify acoustic output parameters such as described above to reduce the size of the cavitation bubbles, and reduce the potential for gas build-up.

For example, as shown in FIG. 2, in certain energy control algorithms, it is desired that as the frequency of the pulse delivery increases, the acoustic energy level for each of the energy pulses would decrease. Similarly, as the acoustic energy level for each of the pulses increases, the pulse frequency would see a corresponding decrease. In particular, FIG. 2 graphically illustrates a relationship between acoustic energy level (of each energy pulse) and pulse frequency that can be incorporated into an embodiment of the energy control algorithm. More specifically, as shown, in this embodiment of the energy control algorithm, as implemented by the system controller, there is a negative linear relationship between the acoustic energy level and the pulse frequency.

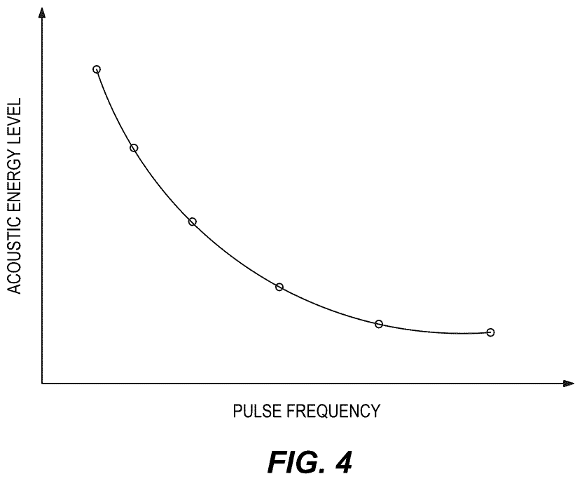

In other embodiments of the energy control algorithm, there can be a negative non-linear relationship between the acoustic energy level and the pulse frequency. It is appreciated that in such other embodiments, as the pulse frequency increases, the acoustic energy level (per pulse) can decrease at an increasing (faster) rate (such as illustrated in FIG. 3), or the acoustic energy level (per pulse) can decrease at a decreasing (slower) rate (such as illustrated in FIG. 4).

In various embodiments, the system controller can implement energy control algorithms that incorporate acoustic energy levels for each pulse that range between approximately 0.05 MPa and 8.0 MPa, and pulse frequencies that range between approximately 0.3 Hz to 15.0 Hz. In such embodiments that have a negative relationship between the acoustic energy level and the pulse frequency, such as shown in FIG. 2, it is appreciated that (i) if the pulse frequency is at or near its minimum of 0.3 Hz, then the acoustic energy level per pulse will generally be at or near its maximum of 8.0 MPa, and (ii) if the pulse frequency is at or near its maximum of 15.0 Hz, then the acoustic energy level per pulse will generally be at or near its minimum of 0.05 MPa. Alternatively, the system controller can implement energy control algorithms with acoustic energy levels for each pulse and pulse frequencies that are higher or lower than the above-noted ranges.

Still alternatively, however, it is appreciated that the system controller can also implement energy control algorithms that exhibit a different relationship between the acoustic energy level of the energy pulses versus the pulse frequency of the energy pulses. In particular, any given energy control algorithm that is implemented by the system controller can incorporate one or more of the following, (1) the acoustic energy level can increase over time, while there is a corresponding increase in the pulse frequency (at a constant (linear), increasing, or decreasing rate); (2) the acoustic energy level can increase over time, while there is a corresponding decrease in the pulse frequency (at a constant (linear, such as shown in FIG. 2), increasing, or decreasing rate); (3) the acoustic energy level can increase over time, while the pulse frequency remains relatively constant; (4) the acoustic energy level can decrease over time, while there is a corresponding decrease in pulse frequency (at a constant (linear), increasing, or decreasing rate); (5) the acoustic energy level can decrease over time, while there is a corresponding increase in pulse frequency (at a constant (linear, such as shown in FIG. 2), increasing, or decreasing rate); (6) the acoustic energy level can decrease over time, while the pulse frequency remains relatively constant; (7) the acoustic energy level can remain relatively constant over time, while the pulse frequency increases over time; (8) the acoustic energy level can remain relatively constant over time, while the pulse frequency decreases over time; and/or (9) the acoustic energy level can remain relatively constant over time, while the pulse frequency also remains relatively constant. It is further appreciated that any given energy control algorithm implemented by the system controller can include any combination of the noted possibilities that are implemented in any possible sequence.

FIG. 3 is a graphical illustration of another relationship between acoustic energy level and pulse frequency that can be incorporated into one or more energy control algorithms having features of the present invention. Somewhat similar to what is shown in FIG. 2, as the frequency of the pulse delivery increases, the acoustic energy level for each of the energy pulses would decrease; and/or as the acoustic energy level for each of the pulses increases, the frequency of the pulse delivery would decrease. However, as opposed to the relationship illustrated in FIG. 2, FIG. 3 illustrates an embodiment of the energy control algorithm wherein there is a negative, but non-linear, relationship between the acoustic energy level and the pulse frequency. More specifically, as shown, as the pulse frequency increases, the acoustic energy level (per pulse) decreases at an increasing rate.

FIG. 4 is a graphical illustration of still another relationship between acoustic energy level and pulse frequency that can be incorporated into one or more energy control algorithms having features of the present invention. Again, somewhat similar to what is shown in FIG. 2, as the frequency of the pulse delivery increases, the acoustic energy level for each of the energy pulses would decrease; and/or as the acoustic energy level for each of the pulses increases, the frequency of the pulse delivery would decrease. However, as opposed to the relationship illustrated in FIG. 2, FIG. 4 again illustrates an embodiment of the energy control algorithm wherein there is a negative, but non-linear, relationship between the acoustic energy level and the pulse frequency. More specifically, as shown, as the pulse frequency increases, the acoustic energy level (per pulse) decreases at a decreasing rate.

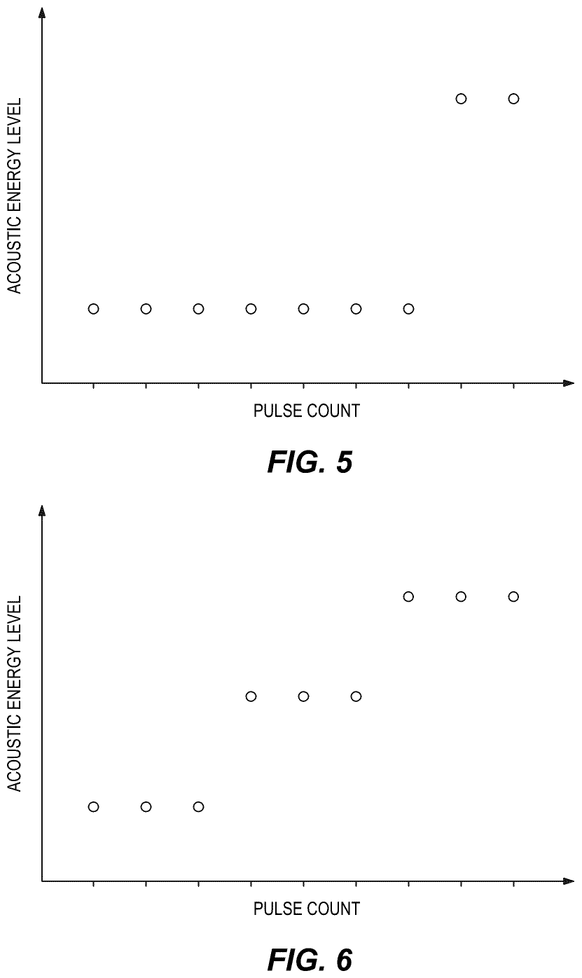

FIG. 5 is a graphical illustration of a relationship between acoustic energy level and pulse count that can be incorporated into one or more energy control algorithms having features of the present invention. As shown in FIG. 5, the relationship between the acoustic energy level and pulse count is such that the energy control algorithm is configured such that the last acoustic shot(s) (or energy pulse(s)) of a treatment cycle has (have) significantly larger acoustic wave energy than the preceding acoustic shots. Stated in another manner, in this embodiment, the relationship between the acoustic energy level and pulse count within this treatment cycle is such that the acoustic energy level of a final energy pulse at an end of the treatment cycle is greater than the acoustic energy level of a first energy pulse at a beginning of the treatment cycle. With such an energy control algorithm, since gas build-up with the last energy pulse(s) is less of a concern, then this could allow for better outcomes with the ability to break up the calcium within the vessel wall at the treatment site.

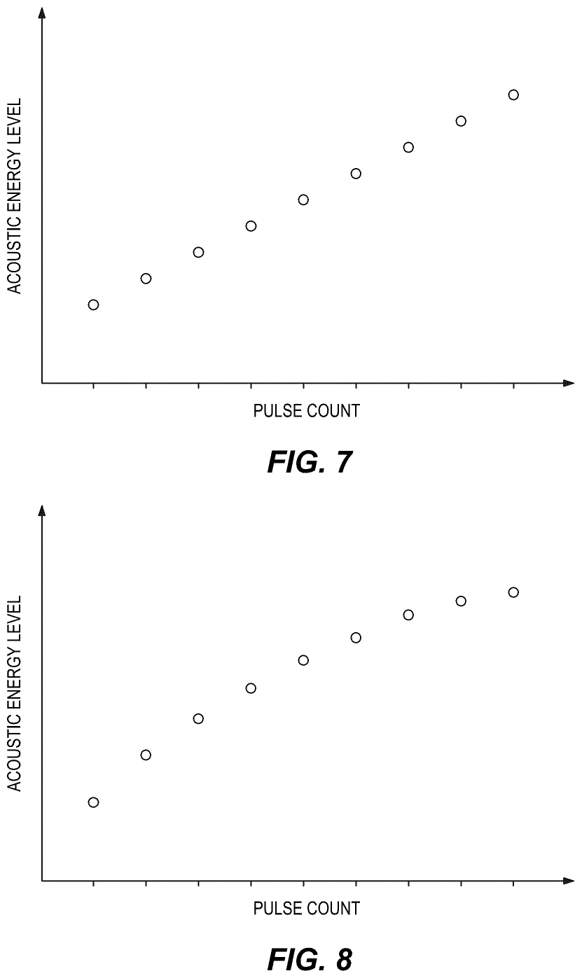

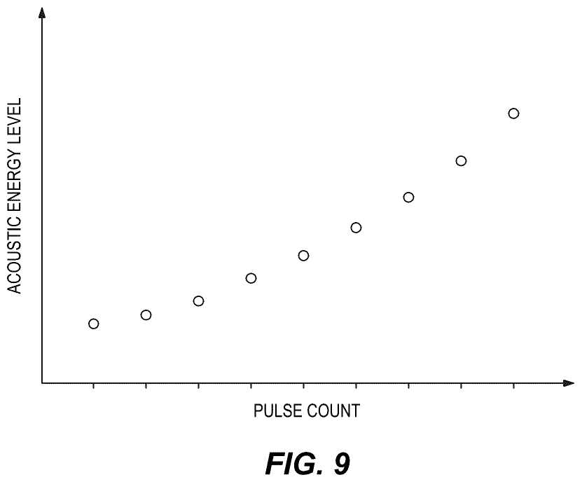

In certain non-exclusive alternative embodiments, the acoustic energy level of each energy pulse can still generally increase as the pulse count increases for a particular treatment cycle, but in a somewhat different manner than what is illustrated in FIG. 5. For example, in one such alternative embodiment, the acoustic energy level of each energy pulse can increase in a stepped manner as the pulse count increases for a particular treatment cycle (with one or more energy pulses at each step, such as shown in FIG. 6). In another such alternative embodiment, the acoustic energy level of each energy pulse can increase in a linear manner as the pulse count increases for a particular treatment cycle (such as shown in FIG. 7). In still another such alternative embodiment, the acoustic energy level of each energy pulse can increase in a non-linear manner, at a decreasing rate, as the pulse count increases for a particular treatment cycle (such as shown in FIG. 8). In yet another such alternative embodiment, the acoustic energy level of each energy pulse can increase in a non-linear manner, at an increasing rate, as the pulse count increases for a particular treatment cycle (such as shown in FIG. 9).

In various embodiments, the system controller can implement energy control algorithms that incorporate acoustic energy levels for each pulse that range between approximately 0.05 MPa and 8.0 MPa, and treatment cycles that range between approximately 10 seconds and 60 seconds. Alternatively, the system controller can implement energy control algorithms with acoustic energy levels for each pulse and treatment cycles that are higher or lower than the above-noted ranges.

Still alternatively, however, it is appreciated that the acoustic energy level of the energy pulses can also exhibit a different pattern over time, such as the acoustic energy level remaining relatively constant over time and/or the acoustic energy level generally decreasing over time. It is further appreciated that the possibilities of the acoustic energy level increasing, decreasing, or remaining relatively constant over time can be combined in any suitable manner within the energy control algorithms.

FIG. 6 is a graphical illustration of another relationship between acoustic energy level and pulse count that can be incorporated into one or more energy control algorithms having features of the present invention. In particular, FIG. 6 illustrates another embodiment of the energy control algorithm that can be configured to have a generally increasing acoustic energy level as the pulse count increases for a particular treatment cycle to deliver increasingly more therapy over the course of a treatment cycle. More specifically, in this embodiment, the acoustic energy level is increasing in a stepped manner, with one or more energy pulses (three as shown in FIG. 6) occurring at each individual acoustic energy level. As with the embodiment of FIG. 5, since gas build-up with the later energy pulses is less of a concern, this could again allow for better outcomes with the ability to break up the calcium within the vessel wall at the treatment site.

FIG. 7 is a graphical illustration of still another relationship between acoustic energy level and pulse count that can be incorporated into one or more energy control algorithms having features of the present invention. In particular, FIG. 7 illustrates still another embodiment of the energy control algorithm that can be configured to have a slow ramp of energy (linear in this embodiment) to deliver increasingly more therapy over the course of a treatment cycle. As with the preceding embodiments, since gas build-up with the later energy pulses is less of a concern, this could again allow for better outcomes with the ability to break up the calcium within the vessel wall at the treatment site.

FIG. 8 is a graphical illustration of yet another relationship between acoustic energy level and pulse count that can be incorporated into one or more energy control algorithms having features of the present invention. In particular, FIG. 8 illustrates yet another embodiment of the energy control algorithm that can be configured to have a slow ramp of energy (non-linear, and at a decreasing rate in this embodiment) to deliver increasingly more therapy over the course of a treatment cycle. As with the previous embodiments, since gas build-up with the later energy pulses is less of a concern, then this could again allow for better outcomes with the ability to break up the calcium within the vessel wall at the treatment site.

FIG. 9 is a graphical illustration of still yet another relationship between acoustic energy level and pulse count that can be incorporated into one or more energy control algorithms having features of the present invention. In particular, FIG. 9 illustrates still yet another embodiment of the energy control algorithm that can be configured to have a slow ramp of energy (non-linear, and at an increasing rate in this embodiment) to deliver increasingly more therapy over the course of a treatment cycle. As with the embodiment of FIG. 5, since gas build-up with the later energy pulses is less of a concern, then this could again allow for better outcomes with the ability to break up the calcium within the vessel wall at the treatment site.

Thus, the various energy control algorithms described herein, and as implemented by the system controller, can include any suitable relationship between the acoustic energy level versus pulse frequency of the energy pulses, as well as any particular control of the acoustic energy level over time (in subsequent pulses within a given treatment cycle).

As noted above, it is appreciated that any of the specifically described energy control algorithms can further incorporate strategies for controlling one or more of pulse width, pulse rise time, overall acoustic energy per treatment cycle, and number of pulses per treatment cycle, with any such parameters being controlled by the system controller to increase, decrease and/or remain relatively constant over time.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the detailed description provided herein. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter system, the system controller, and/or the energy control algorithm implemented therein have been illustrated and described herein, one or more features of any one embodiment can be combined with one or more features of one or more of the other embodiments, provided that such combination satisfies the intent of the present invention.

While a number of exemplary aspects and embodiments of the catheter system, the system controller, and/or the energy control algorithm implemented therein have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

1. A catheter system for treating a treatment site within or adjacent to a vessel wall of a blood vessel, or within or adjacent to a heart valve, the catheter system comprising:
an energy source that generates a plurality of energy pulses;
an energy guide that receives the plurality of energy pulses from the energy source, the energy guide guiding the plurality of energy pulses from a guide proximal end to a guide distal end of the energy guide;
a balloon including a balloon wall that defines a balloon interior, the balloon being configured to retain a catheter fluid within the balloon interior, the guide distal end of the energy guide being positioned within the balloon interior; and
a system controller including a processor that controls the energy source, the system controller being configured to implement an energy control algorithm to control both of (i) a pulse frequency of the plurality of energy pulses, and (ii) an energy level for each of the plurality of energy pulses that are generated by the energy source;
wherein as the pulse frequency of the plurality of energy pulses increases, the energy level for each of the plurality of energy pulses decreases.

2. The catheter system of claim 1 wherein implementation of the energy control algorithm enables the system controller to automatically control both of (i) the pulse frequency of the plurality of energy pulses, and (ii) the energy level for each of the plurality of energy pulses that are generated by the energy source.

3. The catheter system of claim 1 wherein there is a negative linear relationship between the energy level for each of the plurality of energy pulses and the pulse frequency of the plurality of energy pulses.

4. The catheter system of claim 1 wherein there is a negative non-linear relationship between the energy level for each of the plurality of energy pulses and the pulse frequency of the plurality of energy pulses.

5. The catheter system of claim 1 wherein the energy level of each of the plurality of energy pulses that are generated by the energy source are between approximately 0.05 MPa and 8.0 MPa.

6. The catheter system of claim 1 wherein the pulse frequency of the plurality of energy pulses that are generated by the energy source are between approximately 0.3 Hz and 15.0 Hz.

7. The catheter system of claim 1 wherein a treatment cycle is defined by a specified number of energy pulses being generated by the energy source over a specified period of time; and wherein the energy level of a final energy pulse at an end of the treatment cycle is greater than the energy level of a first energy pulse at a beginning of the treatment cycle.

8. The catheter system of claim 7 wherein the energy control algorithm implemented by the system controller further enables the control system to control at least one of (i) a pulse width of each of the plurality of energy pulses, (ii) a pulse rise time for each of the plurality of energy pulses, (iii) overall acoustic energy per treatment cycle, and (iv) a number of pulses per treatment cycle.

9. The catheter system of claim 8 wherein the energy control algorithm implemented by the system controller further enables the control system to control each of (i) the pulse width of each of the plurality of energy pulses, (ii) the pulse rise time for each of the plurality of energy pulses, (iii) the overall acoustic energy per treatment cycle, and (iv) the number of pulses per treatment cycle.

10. (canceled)

11. The catheter system of claim 1 further comprising a catheter shaft, the balloon being coupled to the catheter shaft, and wherein the balloon is selectively inflated with the catheter fluid to expand to an inflated state, the balloon wall being positionable adjacent to the treatment site when the balloon is in the inflated state.

12. The catheter system of claim 11 further comprising a plasma generator that is positioned near the guide distal end of the energy guide, the plurality of energy pulses that are received by the energy guide being emitted from the guide distal end and contacting the plasma generator so that plasma is generated in the catheter fluid retained within the balloon interior; and wherein the plasma generation causes bubble formation that generates a pressure wave that imparts pressure adjacent to the treatment site.

13. The catheter system of claim 1 wherein the energy source is a light source that generates a plurality of light energy pulses; and wherein the energy guide is an optical fiber.

14. A catheter system for treating a treatment site within or adjacent to a vessel wall of a blood vessel, or within or adjacent to a heart valve, the catheter system comprising:
an energy source that generates a plurality of energy pulses;
an energy guide that receives the plurality of energy pulses from the energy source, the energy guide guiding the plurality of energy pulses from a guide proximal end to a guide distal end of the energy guide;
a balloon including a balloon wall that defines a balloon interior, the balloon being configured to retain a catheter fluid within the balloon interior, the guide distal end of the energy guide being positioned within the balloon interior; and
a system controller including a processor that controls the energy source, the system controller being configured to implement an energy control algorithm to control both of (i) a pulse frequency of the plurality of energy pulses, and (ii) an energy level for each of the plurality of energy pulses that are generated by the energy source;
wherein a treatment cycle is defined by a specified number of energy pulses being generated by the energy source over a specified period of time; and
wherein the energy level of a final energy pulse at an end of the treatment cycle is greater than the energy level of a first energy pulse at a beginning of the treatment cycle.

15. The catheter system of claim 14 wherein implementation of the energy control algorithm enables the system controller to automatically control both of (i) the pulse frequency of the plurality of energy pulses, and (ii) the energy level for each of the plurality of energy pulses that are generated by the energy source.

16. The catheter system of claim 14 wherein the energy level of the plurality energy pulses increases in one of a stepped manner, a linear manner, and a non-linear manner from the first energy pulse to the last energy pulse of the treatment cycle.

17. The catheter system of claim 14 wherein the treatment cycle lasts between approximately 10 seconds and 60 seconds.

18. The catheter system of claim 14 wherein the energy control algorithm implemented by the system controller further enables the control system to control at least one of (i) a pulse width of each of the plurality of energy pulses, (ii) a pulse rise time for each of the plurality of energy pulses, (iii) overall acoustic energy per treatment cycle, and (iv) a number of pulses per treatment cycle.

19. The catheter system of claim 18 wherein the energy control algorithm implemented by the system controller further enables the control system to control each of (i) the pulse width of each of the plurality of energy pulses, (ii) the pulse rise time for each of the plurality of energy pulses, (iii) the overall acoustic energy per treatment cycle, and (iv) the number of pulses per treatment cycle.

20. A catheter system for treating a treatment site within or adjacent to a vessel wall of a blood vessel, or within or adjacent to a heart valve, the catheter system comprising:
an energy source that generates a plurality of energy pulses;
an energy guide that receives the plurality of energy pulses from the energy source, the energy guide guiding the plurality of energy pulses from a guide proximal end to a guide distal end of the energy guide; and
a system controller including a processor that controls the energy source, the system controller being configured to implement an energy control algorithm to control both of (i) a pulse frequency of the plurality of energy pulses, and (ii) an energy level for each of the plurality of energy pulses that are generated by the energy source;
wherein as the pulse frequency of the plurality of energy pulses increases, the energy level for each of the plurality of energy pulses decreases;
wherein a treatment cycle is defined by a specified number of energy pulses being generated by the energy source over a specified period of time;
wherein the energy level of a final energy pulse at an end of the treatment cycle is greater than the energy level of a first energy pulse at a beginning of the treatment cycle; and
wherein the energy control algorithm implemented by the system controller further enables the control system to control each of (i) a pulse width of each of the plurality of energy pulses, (ii) a pulse rise time for each of the plurality of energy pulses, (iii) overall acoustic energy per treatment cycle, and (iv) a number of pulses per treatment cycle.

21. The catheter system of claim 20 further comprising a balloon including a balloon wall that defines a balloon interior, the balloon being configured to retain a catheter fluid within the balloon interior, the guide distal end of the energy guide being positioned within the balloon interior.
